(19) United States
(12) Patent Application Publication
(10) Pub. No.: US 2001/0043916 A1
(43) Pub. Date: Nov. 22, 2001

(54) Method using filtration aids for the separation of virus vectors from nucleic acids and other cellular contaminants

(76) Inventors: David S. McNeilly, Norforlk, MA (US); William O. Osburn, Brighton, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 09/742,247

(22) Filed: Dec. 20, 2000

Related U.S. Application Data
(60) Provisional application No. 60/173,584, filed on Dec. 29, 1999.

(51) Int. Cl. C12N 7/02

(52) U.S. Cl. 424/93.6; 435/239

(57) ABSTRACT

Methods are disclosed for the purification of encapsulated viruses. The methods are advantageous in that they employ filtration aids, together with low concentrations of metal ions, in place of nucleases for purification. This provides important advantages for commercial scale purification of viruses.

### FIELD OF THE INVENTION

The present invention relates to a non-enzymatic method for separating virus particles in cell lysates from genomic DNA, RNA and other host-cell components by using diatomaceous (silicious) earth or other filtration aid, such that the subsequent purification of viruses (particularly encapsulated viral vectors) using filtration and either centrifugation in density gradients or column chromatography can proceed with minimal interference from the host-cell, contaminating constituents.

### BACKGROUND OF THE INVENTION

Following the release of virus(es) from infected (i.e., host) cells, removal of host-cell DNA and RNA has been a critical step for improving the operation and efficiency of further (i.e., “downstream”) steps used during the manufacture (i.e., purification) of encapsulated viruses. This is particularly important for those viruses that are intended for clinical use in animals (including humans). Examples of (but not restricted to) these viruses are adenovirus serotypes (or strains) 2 and 5 and adeno-associated viruses (here abbreviated as Ad2 and Ad5, and AAV respectively).

Currently the method most frequently used for the elimination of macromolecular nucleic acids (i.e., DNA and RNA) during the purification of viruses employs a variety of hydrolyzing nuclease enzymes. These are collectively termed nucleases and are most often used at an early, “upstream” step in the purification scheme. For this there are several commercially available nucleases and nuclease mixtures (“cocktails”). The purpose of using such enzyme preparations is for the hydrolysis or digestion of the host cell DNA and RNA. After hydrolysis the smaller-sized nucleic acids are either removed (for example, by filtration) or continued through the purification to be to separated from viruses by some other physical difference (for example, by exploiting differences in buoyant densities during ultracentrifugal sedimentation in a cesium chloride, CsCl, density gradients).

All of the host cell components and any additives (such as nucleases) have the unfortunate potential of being capable of co-purifying with the viruses into the final, purified composition. During the processing of the cell lysate, the additives and host-cell's chromosomal (or genomic) DNA, RNA, protein, membranes and other cellular debris components can interfere with equilibrium sedimentation of the viruses during, for example, the conventional CsCl density gradient centrifugation method. In alternative procedures, such as chromatography, the contaminants can and do compete with the viruses for interactions with chromatographic media. The latter is particularly true when attempting to use the positively charged, anion exchange chromatography resins in the purification of the viruses. Singular, among the contaminating components, and characterized by its large molecular size and viscosity, is the host's genomic DNA that is released by the lysis procedure. If not hydrolyzed (or otherwise removed) this poly-anionic nucleic acid frequently causes the fouling of filtration and chromatographic equipment.

In addition to the operational and logistical problems associated with current virus purification procedures, the use of nucleases presents other significant disadvantages. Examples of these disadvantages include the following:

a) Purity and lot variability of commercially available preparations of nucleases has been observed. The nuclease reagent that is added must be sufficiently pure to avoid damage to the virus protein-containing encapsulation. This damage can be caused by such potential contaminants as any one of a large variety of protein degrading enzymes (proteinases or proteases). Lot variability can also be the result of decreases or increases in the activities of nucleases. It was observed, for example, that the loss of a virus preparation was due, in part, to an unusually active lot of commercially available nuclease.

b) Nucleases (that are commercially available and specifically produced for processing biological materials for commercial or clinical use) are expensive and contribute the principle, bill-of-materials cost to most virus production operations and,

c) The addition of any reagent (in this instance, addition of the nuclease) to virus purification, necessitates the need for the development or incorporation of Quality Control assays designed to demonstrate the absence of that particular reagent in the final product. Elimination of a reagent (such as nuclease) from the process eliminates the need to assay for presence of that reagent, and simplifies product specifications.

In part as a result of investigations into appropriate methods for the isolation of “naked” plasmid DNA in an Eschericia coli (E. coli) expression system, it was found that, under appropriate conditions, the bacterial DNA adsorbed to certain grades of diatomaceous earth. Diatomaceous earth is found in mineral beds throughout the world and is known to be composed of the silicon dioxide (SiO₂) skeletons of extinct marine organisms, diatoms. Silicon dioxide is the major constituent in quartz sand (and therefore, glass). It is inert and is further known to adsorb nucleic acids under certain appropriate conditions including in the presence of ammonium sulfate or any chaotropic salt. It does this by creating salt bridges between the hydroxyl of the silicate and the negatively charged phosphates on nucleic acids.

It was reasoned by the inventors that DNA, possibly RNA and other host-cell constituents, also found in animal cells used for the expression of viruses, could be removed if those contaminants interacted with the diatomaceous earth. Indeed, when translated into the animal cell system for the expression of viruses, it was found that diatomaceous earth did, in fact, adsorb nucleic acids that absorb UV light at 260 nm (A₂₆₀). And this occurred whether or not the cells were infected with virus. It was further found that purified virus particles did not adsorb to (nor become entrapped in) diatomaceous earth. Nearly 100% of them could be recovered in filtrates or supernatants of filtered or centrifuged suspensions of virus and diatomaceous earth.

Thus, under defined conditions, the incorporation of diatomaceous earth or other suitable filtration aid early in virus purifications (i.e., soon after host-cell lysis) could eliminate virtually all of the genomic DNA and RNA, most (if not all) of the cellular debris (such as membranes and membrane-associated molecules) and some proteins, without the use of Benzonase™ or other nuclease enzymes. The idea also provided further justification for the use of the inert diatomaceous earth. That is, the chemically defined, biologically inert diatomaceous earth (in place of the biological-source nucleases) should improve the virus product and would eliminate specification tests that would have to be designed to demonstrate the removal (i.e., absence) of the nuclease (or nucleases) in the final product.

### SUMMARY OF THE INVENTION

In the art of virus production a variety of nucleic acid hydrolyzing enzymes (collectively termed nucleases) is most frequently used to hydrolyze (in other words, “digest” or “break down”) DNA into smaller (or lower molecular weight) fragments during the early steps in the purification. The method of this invention specifically eliminates the need for employing such nucleases by using, instead, controlled amounts of a suitable commercially available, chemically defined and inert, filter aid; specifically, diatomaceous earth, Whatman™ CDR [Cell Debris Remover] material or DEAE-Cellulose. By this method, host-derived DNA, RNA, cell debris and some protein are physically removed. Eliminating the need for exogenous nucleases also eliminates the requirement to test for them, improves the quality of the virus product and increases process yields by improving the capabilities of such downstream operations as filtration and chromatography.

Accordingly, a purification method, suitable for the purification of virus, particularly encapsulated viruses, such as adenovirus, adeno-associated virus, retroviruses, including lentiviruses, and alphaviruses, wherein a suitable filtration aid such as DE is substituted for nuclease enzymes, is proposed (FIG. 1). The method preferably comprises at least two steps. The first step comprises treating a virus containing cell culture or composition with diatomaceous earth or other suitable filtering aid. The second step comprises subjecting the virus containing culture or composition resulting from the DE treatment to further purification steps that either adsorb viruses or the contaminants associated with them. The second purification step preferably comprises chromatography steps that exploit at least two different physical properties interactions between viruses (and contaminants) and available chromatography media. Of course, processes may be used which employ additional purification steps in order to further enhance purity and/or stability of the resulting purified compositions.

An alternative method for removing host cell DNA and RNA without using Benzonase™ or other nuclease enzymes comprises the use of dead end filtration employing filtration aids such as Whatman™ CDR [Cell Debris Remover] material, which functions on an ionic basis. In the case of AAV [which does not bind to DEAE-cellulose], an additional alternative method for removing host cell DNA and RNA without using Benzonase™ comprises using as a filtration aid DEAE-cellulose, such as Whatman™ DEAE-cellulose. In order to utilize DEAE-Cellulose with adenovirus and other viruses which may bind to DEAE-cellulose, one must adjust the conditions of ionic strength, etc. so that the virus either readily elutes or does not bind to the DEAE-cellulose. Similar to the use of DE for purification of virus, these alternative systems have the advantage of accomplishing the removal of host cell DNA and RNA without the need for Benzonase™ or other nuclease enzymes in the purification process. Thus, in another embodiment, the present invention comprises a method for removal of host cell DNA and RNA from a composition containing encapsulated viruses without the use of nuclease enzymes, comprising comprises treating a virus-containing cell culture or composition with a filtration aid, such as diatomaceous earth, Whatman™ CDR or DEAE-cellulose, and one or more additional purification steps that either adsorb viruses or the contaminants associated with them.

In preferred embodiments, a metal ion, including monovalent ions, such as potassium [K⁺] or sodium [Na⁺], and more preferably, divalent ion, such as nickel [Ni⁺²], zinc [Zn⁺²], barium [Ba⁺²], cobalt [Co⁺²], magnesium [Mg⁺²], manganese [Mn⁺²], calcium [Ca⁺²], or trivalent ion, such as ferric iron [Fe⁺³] is used during filtration to promote maximal DNA and RNA binding. The metal ion may be added in the form of a salt, for example, zinc acetate or nickel chloride. Other forms of salt may be useful in the present invention, including chlorides, acetates, citrates, phosphates and sulfates.

Accordingly, the present invention comprises methods for purification of encapsulated viruses from cell culture. In preferred embodiments, the methods of the present invention comprise:

(a) lysing a cell culture containing encapsulated virus;

(b) subjecting the composition resulting from step (a) to filtration with a substance selected from the group consisting of Diatomaceous Earth (DE) and poly-anionic cellulose filter aids (e.g., Whatman™ CDR or DEAE Cellulose), to generate a filtrate;

(c) subjecting the filtrate of step (b) to one or more suitable concentration and diafiltration steps to generate concentrated and diafiltered retentate; and

(d) subjecting the concentrated or diafiltered retentate of step (c) to one or more suitable purification steps and collecting a purified composition containing encapsulated viruses.

In preferred embodiments, virus-containing cells may optionally be harvested from cell culture broth, using, for example, tangential flow filtration or centrifugation prior to cell lysis. Alternatively, cell lysis may be performed directly on cell culture containing unharvested cells. In preferred embodiments, cell lysis may be accomplished by microfluidization, treatment with detergent with or without a static mixer, or subjecting to freeze/thaw cycles. Cell lysis may also be accomplished by any means known in the art.

A main feature of the present invention is in the use of filtration aids as described in step (b). This step may use diatomaceous earth, poly-anionic cellulose cellulose [or other poly-anionic vehicles] based filtration cellulose to improve the separation of viruses from DNA and RNA species present in the cell culture. The filtration step preferably includes the presence of small amounts of a metal ion or salt. The metal ion or salt may be any metal ion that is suitable for promoting DNA or RNA binding. In preferred embodiments, the metal ion is provided by use of a salt selected from the group consisting of zinc chloride, zinc acetate, nickel chloride, nickel sulfate, ferric chloride, copper chloride and barium chloride. Prior art methods often have used digestive enzymes, such as Benzonase™, to degrade such DNA and RNA species. However, as described above, such methods have serious disadvantages.

Subsequent to filtration, the method of the present invention comprises one or more concentration and/or diafiltration steps, and purification steps. Methods for concentration, diafiltration and purification are well-known in the art, and the skilled artisan may select the optimal combination of such steps. Such optimization is contemplated, and does not vary from virus. In certain preferred embodiments, the filtration aid is a poly-anionic cellulose based filtration aid, and salt concentration is adjusted so that host cell nucleotides bind to poly-anionic celluloses and virus flows through the filter into the filtrate.

In still other embodiments of the present invention, the methods of the invention comprise loading the filtrate from step (b) into a device used for concentration of biological molecules. In other embodiments, the method comprises employing a dialysis or buffer-exchange device which device comprises a membrane having a pore size suitable for retaining virus particles. In other embodiments, the methods of the present invention may further comprise concentrating the retained virus particles are concentrated in solution by ultrafiltration.

In other embodiments, step (c) may comprise one of the following: (1) employing a dialysis or buffer-exchange device which device comprises a resin having a pore size capable of separating the virus particles from larger and smaller molecular size contaminants; (2) employing a concentration device which device comprises a membrane pore size suitable for the passage of materials containing molecular sizes smaller than virus particles; (3) dialyzing or buffer-exchanging the composition containing virus particles prior to concentration; (4) concentrating the composition containing virus particles prior to diafiltration.

In further preferred embodiments, the diafiltration step of step (c) produces a diafiltered, non-concentrated virus particles suitable for loading onto a suitable anion exchange chromatography resin, a suitable hydrophobic interaction chromatography resin to generate a flow-through pool, a suitable pseudo-affinity resin, or a suitable cation exchange chromatography resin.

In other preferred embodiments, the concentrated and diafiltered retentate of step (c) is suitable for mixing the concentrated virus particles with cesium chloride; or for loading the concentrated virus particles onto (and promoting their adsorption to) a suitable anion exchange chromatography resin.

In other preferred embodiments, the concentration step of step (c) produces a is concentrated, non-diafiltered virus particles suitable for loading onto a suitable hydrophobic interaction chromatography resin to generate a flow-through pool; or for loading onto (and promoting their adsorption to) a suitable cation exchange chromatography resin.

With respect to the purification steps of step (d), a vast number of permutations and combinations of one or more purification steps, are possible for treating compositions containing encapsulated viruses, including: (1) adsorbing the encapsulated virus to a suitable anion exchange chromatography column; (2) adsorbing the encapsulated virus onto a suitable pseudo-the present invention. It is important to note that the concentration and diafiltration steps of step (c) and the purificaton steps of step (d) may take place iteratively, as described further herein. Hence the retentate of step (b) may be subjected to a concentration step, resulting in a composition containing concentrated, non-diafiltered virus particles. This composition may be subjected to one or more purification steps of step (d). The resulting composition containing purified and concentrated virus particles may be subjected to one or more diafiltration steps, which may be followed by further purification steps. Analogously, the unconcentrated filtrate of step (b) may be subjected to one or more diafiltration steps. The composition containing diafiltered, non-concentrated virus particles may then be subjected to one or more purification steps of step (d). The resulting composition containing purified and diafiltered virus particles may be subjected to one or more concentration steps, which may be followed by further purification steps.

The diafiltration step(s) of the invention preferably may comprise subjecting the retentate to dialysis (buffer-exchange), using tangential flow filtration. In certain preferred embodiments, one or more diafiltration steps, using tangential flow filtration, may optionally be employed prior to the filter aid mediated filtration steps of step (b). In these methods, one or more diafiltration steps, using tangential flow filtration are still desirable to be performed subsequent to step (b).

In preferred embodiments, the method comprises the use of optimal concentrations of metal ion salts during DE or poly-anionic cellulose cellulose [or other poly-anionic vehicles] based filtration cellulose to promote maximal DNA and RNA binding. The metal ion or salt may be any metal ion that is suitable for promoting DNA or RNA binding, but preferably is selected from the group consisting of Zn, Ni, Cu, Ba, Mg, Mn, Co, K or Na, such as zinc chloride, zinc acetate, nickel chloride, nickel sulfate, ferric chloride, copper chloride, barium chloride, magnesium chloride, manganese chloride, sodium chloride, sodium phosphate, sodium acetate, potassium chloride, potassium phosphate and potassium acetate.

In other embodiments, the metal ion or salt may include other metals, such as potassium, magnesium, sodium, cobalt, and manganese and other salt forms, such as chlorides, acetates, sulfates, citrates and phosphates. For certain metal ion salts, such as sodium chloride and magnesium chloride, it is preferred to have present trace amounts of another metal ion, preferably zinc. In preferred methods, optimal concentrations of metal ion salts during DE or poly-anionic cellulose [or other poly-anionic vehicles] filtration are used to promote maximal DNA and/or RNA binding and may further comprise addition of histidine, imidazole or another amino acid that can modify the binding of metal ions to either the host cell nucleotides or to the affinity resin; (3) loading the flow-through pool onto a suitable cation exchange resin; (4) mixing the encapsulated viruses with cesium chloride and subjecting the mixture to ultracentrifugation; (5) loading the encapsulated viruses onto a suitable hydrophobic interaction chromatography resin under conditions to generate a flow-through pool and collecting a purified composition containing encapsulated viruses in the flow-through pool; and combinations of the above.

Thus, for example, in certain preferred embodiments of the invention, the purification steps of step (d) comprise mixing the concentrated virus particles with cesium chloride and subjecting the mixture to ultracentrifugation. In others, the purification steps may comprise first loading the composition containing diafiltered, non-concentrated virus particles onto and adsorbing the encapsulated virus to a suitable anion exchange chromatography column and using suitable elution to collect a purified composition containing encapsulated viruses. In other preferred embodiments, the purification steps comprise loading the concentrated and diafiltered retentate of step (c) containing encapsulated viruses onto a suitable anion exchange chromatography resin to produce a purified composition containing encapsulated viruses, followed by a second purification step of loading the purified composition containing encapsulated viruses onto a suitable hydrophobic interaction chromatography resin under conditions to generate a flow-through pool and collecting a purified composition containing encapsulated viruses in the flow-through pool. In yet another preferred embodiment, the purification steps of step (d) comprise loading concentrated, non-diafiltered virus particles onto a suitable hydrophobic interaction chromatography resin to generate a flow-through pool, followed by loading the flow-through pool onto a suitable cation exchange resin and, using suitable elution conditions, and collecting a purified composition containing encapsulated viruses.

In other preferred embodiments, the purification steps of step (d) comprise adsorbing the concentrated and diafiltered retentate of step (c) to a suitable anion exchange chromatography resin and, using suitable elution conditions, collecting a purified composition containing encapsulated viruses. In yet other preferred embodiments, the purification steps of step (d) further comprise loading the purified composition containing encapsulated viruses onto a suitable hydrophobic interaction chromatography column under conditions to generate a flow-through pool and collecting a purified composition containing encapsulated viruses in that flow-through pool. In other embodiments, the purification steps of step (d) further comprise adsorbing the purified composition containing encapsulated viruses from the flow-through pool from a hydrophobic interaction chromatography column to a suitable cation exchange resin and, using suitable elution conditions, collecting a purified composition containing encapsulated viruses.

### BRIEF DESCRIPTION OF THE FIGURES

FIG. 1 is a process flow diagram of certain embodiments of the methods of purification of virus from cells using DE, as described in the present invention.

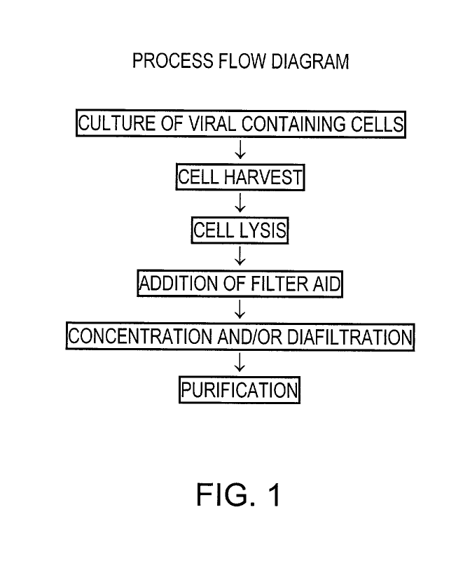

FIG. 2 plots DNA recovery data obtained from DE filtrates where DE filtration was performed in the presence of varying salt concentrations (see Table 1). Estimation of DNA concentration was obtained by use of quantitative PCR. DNA Removal, plotted as percent reduction (% removal, y axis), by DE was determined where percent removal was calculated by the following formula:

% DNA Removal=[(DNA recovered prior to DE Filtration−DNA recovered post DE Filtration)/DNA recovered prior to DE Filtration]×100

FIG. 3 plots DNA recovery data obtained from DE filtrates where DE filtration was performed in the presence of varying MgCl₂ concentration (see Table 2). Estimation of DNA concentration was obtained by use of Qiagen DNA purification tips used as detailed in the manufacturers instructions. DNA Removal, plotted as percent reduction (% removal, y axis), by DE was determined where percent removal was calculated by the following formula:

% DNA Removal=[(DNA recovered prior to DE Filtration−DNA recovered post DE Filtration)/DNA recovered prior to DE Filtration]×100

FIG. 4 plots DNA recovery data obtained from DE filtrates where DE filtration was performed in the presence of varying NaCl concentration (see Table 2). Estimation of DNA concentration was obtained by use of Qiagen DNA purification tips used as detailed in the manufacturers instructions. DNA Removal, plotted as percent reduction (% removal, y axis), by DE was determined where percent removal was calculated by the following formula:

% DNA Removal=[(DNA recovered prior to DE Filtration−DNA recovered post DE Filtration)/DNA recovered prior to DE Filtration]×100

### DETAILED DESCRIPTION OF THE INVENTION

In the following description, the following abbreviations are used:

Ad2—Adenovirus, serotype 2

Ad5—Adenovirus, serotype 5

AEX—anion exchange

CsCl=cesium chloride

CDR—Whatman™ cell debris removal poly-anionic cellulose based filter aid

DE—diatomaceous earth

DEAE—diethylamino ethyl, an anion exchange resin

DF—diafiltration

HFF—hollow fiber filtration

HIC—hydrophobic interaction chromatography

Lysate-cells which have been microfluidized or otherwise disrupted to release viruses.

NMW—nominal molecular weight

PBS—phosphate-buffered saline

PCR—polymerase chain reaction

SEC—size exclusion chromatography

TFF—tangential flow filtration

Tris—2 amino-2(hydroxymethyl)-1,3-propdanediol; Tris(hydroxymethyl)amino-methane

UF—Ultrafiltration

ZnCl₂—zinc chloride

NaCl—sodium chloride

(NH₄)₂SO₄—ammonium sulfate

293 Cells—a line of human embryonic kidney cells

Supporting Procedures:

Cell Harvest:

Viral containing cells are removed from cell culture by decanting or pumping the cell suspension into a suitable container or preferably by first transferring the cells to a suitable container and then concentrating and diafiltering them using a HFF device.

Cell Lysis:

Viral containing cells are lysed by any suitable homogenization method known to the art. Two illustrative methods are:

1) Freeze/Thaw.

Media and cells were poured into appropriately sized centrifuge tubes and frozen by immersion in a dry ice-ethanol bath. After the suspension was completely frozen the centrifuge tubes were then thawed in a 37° C. water bath. This procedure was repeated twice more to ensure complete cell lysis.

The solution was then transferred into an appropriately sized container for further processing or the material was frozen for future use.

2) Microfluidization.

Prior to cell lysis the microfluidizer (Microfluidics model 110, Microfluidics Co, Cambridge Mass., U.S.A.) was primed with an appropriate buffer solution.

Cell containing media was drawn into the microfluidizer from the harvest container using any suitable tubing. The cells are broken by cavitation. The lysate is then collected in any appropriately collection vessel.

Treatment with CDR.

The virus-containing lysate is diluted by addition of an equal volume of a solution containing 10 mM sodium phosphate buffer pH 7.4 containing 10% Glycerol, 0.25% Tween 80. CDR (Cell Debris remover, Whatman™ Biochemicals, Maidstone, England) is then added to the suspension at a ratio of 0.1 g CDR/mL of solution. The combined virus/cell lysate/CDR suspension is then stirred at 4° C. for 30 min. to achieve a uniform suspension. While the suspension is stirring, host cell DNA, RNA and other host cell components are allowed to adsorb to the CDR. The cell debris and CDR are then removed by pumping the suspension through a dead-end Biocap filtration device (CUNO Fluid Purification, Meriden, Conn., USA). (Note: Any type of dead-end or depth filtration device known to the art that allows virus particles to flow through and, at the same time, retains the CDR-DNA complex and other cell-associated solids can be used for this step).

Tangential Flow Filtration (TFF) Procedure: Ultrafiltration (UF) and Diafiltration (DF).

In preparation for subsequent (i.e., downstream) purification steps, the recovered virus-containing filtrate from the depth filtration step is then concentrated by ultrafiltration (UF) using an AG/T UFP 500 C9A TFF device fitted with a membrane having a nominal molecular weight same TFF device, the retained virus particles are dialyzed (buffer-exchanged) by a diafiltration (DF) procedure. (Note: Any type of TFF (or size exclusion chromatography; i.e., SEC) device known to the art that either retains virus particles (TFF) or otherwise separates other contaminating components by size (e.g., SEC) can be used for this step. In addition, the dialysis solution can be any of those that have the capacity to buffer in the range of pH 6 to 8; for example phosphate).

At this stage the virus-enriched, host cell, nucleic acid- and cell debris-depleted suspension is suitable for further virus purification by any of the methods such as cesium chloride [CsCl] density gradient centrifugation or various chromatographies known to the art.

Determination of Virus Infectivity (Titer Assay)

Human 293 cells are cultured in a 37° C. incubator prior to use in the assay. This plate is called the cell plate. Viral samples are serially diluted 1,000,000 fold and then 150 μL of diluted sample are then transferred to 4 wells of a 96 well microtiter plate. The samples are then further serially diluted 1:2 twenty two times. The diluted samples are then transferred to the cell plate and the infected cell plate is then incubated for 72 hours at 37° C.

The transgene present in all vectors used for development purposes (used according to techniques familiar to those knowledgeable in the art) expresses a green fluorescent protein when observed under an inverted fluorescent microscope. Plates are scored for infection (i.e., infectivity units) following immediate transfer of the cell plates from incubator to the microscope. This simple procedure proceeds moreover without the need of any reagents.

Virus Purification

Without limitation, examples of Pseudo-affinity resins appropriate for purification of adenoviruses include Mimetic Blue (1 and 2) A6XL, Mimetic RED (2 and 3) A6XL, Mimetic Orange (1, 2 and 3) A6XL, Mimetic Yellow (1 and 2) A6XL and Mimetic Green A6XL (ProMetic Biosciences, Montreal (Quebec) Canada), and Blue Sepharose CL-6B and Red Sepharose CL-6B (AmershamPharmacia Biotech, Upsala, Sweden). Without limitation, examples of HIC resins appropriate for purification of adenoviruses include EMD phenyl and EMD propyl (EM Separations Technology, Gibbstown, N.J., USA), Phenyl Sepharose and Octyl Sepharose (AmershamPharmacia Biotech, Upsala, Sweden) and TSK ether, TSK butyl and TSK phenyl (TosoHaas, Montgomeryville, Pa., USA). Without limitation, examples of appropriate anion exchange resins include EMD DEAE (EM Separations Technology, Gibbstown, N.J., USA), DEAE Sepharose (AmershamPharmacia Biotech, Upsala, Sweden) and TSK DEAE 650 and TSK DEAE 750 (TosoHaas, Montgomeryville, Pa., USA). Without limitation, examples of appropriate cation exchange resins include: EMD SO₃ and EMD COO (EM Separations Technology, Gibbstown, N.J., USA), CM and S Sepharose (AmershamPharmacia Biotech, Upsala, Sweden) and TSK CM and SP (TosoHaas, Montgomeryville, Pa., USA).

Determination of Protein

Protein concentration of samples was determined using the BCA method (Pierce Chemical Co. Rockford, Ill., USA). The assay (used according to techniques familiar to those knowledgeable in the art) was performed as described in the manufacturers instructions).

DNA Ouantitation

DNA levels contained in samples taken both prior to and after completion of DE filtration were assayed using Roche High Pure PCR Template Preparation Kits (Roche Molecular Biochemicals, Indianapolis, Ind., USA). DNA isolation used according to techniques familiar to those knowledgeable in the art, was performed using the manufacturers instructions. DNA concentrations were estimated by quantitative real-time PCR analysis of isolated DNA using a Lightcycler apparatus (Roche Molecular Biochemicals, Indianapolis, Ind., USA). Primers and a flourimetric probe for the GAPDH (glyceraldehyde-3-phosphate dehydrogenase) gene were designed at Applied Biosystems (Foster City, Calif.) and synthesized by Operon Technologies (Alameda, Calif.). Forty-five PCR cycles were performed. Degradation of the flourimetric probe by Taq polymerase, was analyzed following each cycle. A standard curve was generated using human genomic DNA from Clontech (Palo Alto, Calif.).

RNA Ouantitation

RNA levels contained in samples taken both prior to and after completion of DE filtration were assayed using Roche High Pure RNA Isolation Kits (Roche Molecular Biochemicals, Indianapolis, Ind., USA). RNA isolation used according to techniques familiar to those knowledgeable in the art was performed using the manufacturer's instructions with the exception that DNase digestion occurred prior to loading of the sample on the column instead of after sample was loaded onto column. RNA concentrations were estimated by quantitative real-time RT-PCR analysis of isolated RNA using a Lightcycler apparatus (Roche Molecular Biochemicals, Indianapolis, Ind., USA). Primers and a flourimetric probe for the rRNA cDNA sequence were designed at Applied Biosystems (Foster City, Calif.) and synthesized by Operon Technologies (Alameda, Calif.). A reverse transcription cycle was followed by forty-five cycles of PCR. Degradation of the flourimetric probe by Taq polymerase, was analyzed following each PCR cycle. A standard curve was generated using human total kidney RNA from Clontech (Palo Alto, Calif.). DNA contamination of the RNA samples was determined by real-time PCR analysis of the isolated RNA sample using the above mentioned primers and probe. Forty-five PCR cycles were performed. Degradation of the flourimetric probe by Taq polymerase, was analyzed following each cycle. A standard curve was generated using human genomic DNA from Clontech (Palo Alto, Calif.). The concentration of the contaminating DNA was subtracted from the estimated RNA concentration to determine the real RNA concentration of the sample.

The following examples illustrate practice of one embodiment of the invention, with respect to purification of adenovirus serotype 2 [Ad2] using diatomaceous earth [DE] as the filtration aid. As described above, other filtration aids are available for such purification processes, which the skilled artisan will recognize as advantageous compared with the use of nuclease enzymes. The examples are not limiting in any respect, and the skilled artisan will readily appreciate that many variations, additions and modifications for purification of adenovirus and other viruses, including the use of numerous chromatographic and other purification techniques, are available. Such variations, additions and modifications constitute part of the present invention.

### EXAMPLES

### Example 1

### Optimization of DNA binding to Diatomaceous Earth (DE)

Cell Lysis

After harvest, the suspended cells (line 293), that were infected with the virus, were lysed by a single passage through a (Model 110, Microfluidics Co, Cambridge, Me., USA) microfluidizer. (Note: Although use of a microfluidizer is the preferred method, any method of cell homogenization or lysis can be used).

Optimization of DNA binding to Diatomaceous Earth (DE); Effect of Salts

As determined by PCR assay, both salt composition and salt concentration were found to play a role in binding of DNA and RNA to DE. Experimentally, 25 mL of lysate was diluted with 25 mL of 10 mM sodium phosphates, pH 7.4, containing 10% glycerol and 0.25% Tween-80. The metal salt, such as zinc acetate, zinc chloride, ferric iron chloride, nickel chloride, barium chloride, sodium chloride or magnesium chloride, concentration was adjusted such that the final concentration in each dilution buffer would be 2× the final salt concentration once the viral containing lysate was diluted. (Final salt concentrations tested are detailed in Table 1). When sodium chloride or magnesium chloride are used, it is preferred that trace amounts of another metal ion such as zinc are also present or added. Final MgCl₂ and NaCl concentrations tested are detailed in Table 2. After dilution, 2.5 g of DE was added to the suspension. The suspension was then stirred at room temperature. After 30 min., the suspension was filtered using a 0.45 μm CA filter (Gelman Sciences, Ann Arbor Mich., USA)

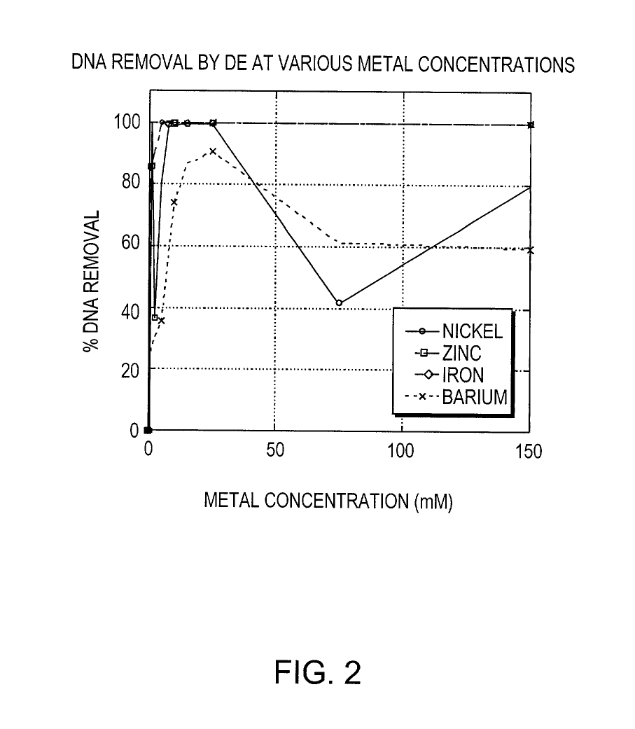

After the experimental procedures were completed, amounts of nucleic acid (DNA and RNA) and virus recovery were determined. To obtain the amount of DNA removed by various metals, samples were assayed as described above. The results are presented in FIG. 2. As can be seen, increasing salt concentrations increase binding of DNA and, presumably, RNA to DE. Zinc and ferric iron appear to require similar salt concentrations for DNA removal. DE binds approximately 100 % of host cell nucleotides in the presence of approximately 1 mM of either salt at pH 7.4. Interestingly, the results indicated that optimal concentrations of barium and nickel may be required for maximal binding of host cell nucleotides to DE.

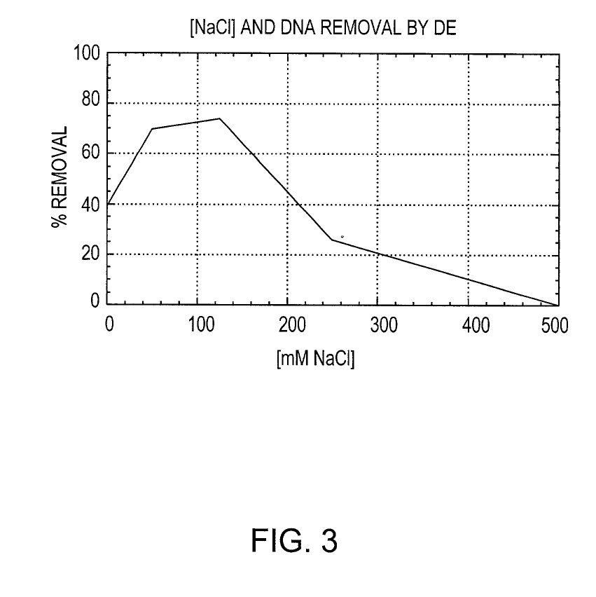

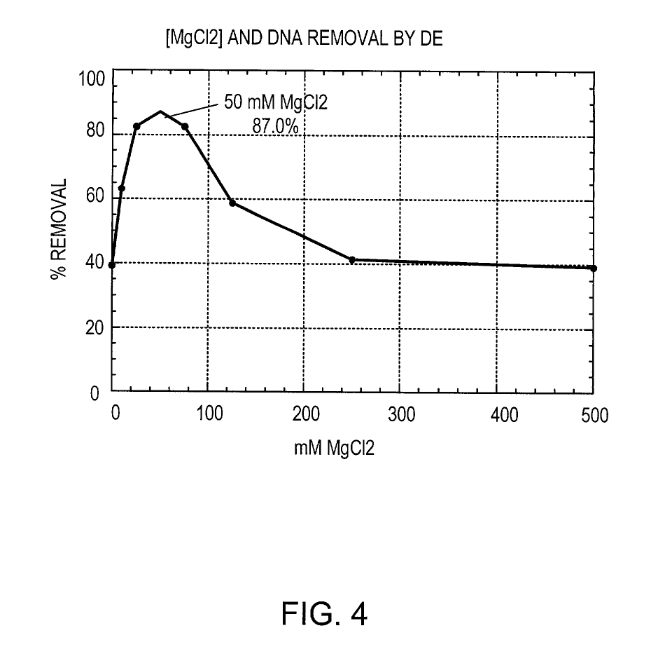

Similar experimentation was conducted with sodium chloride and magnesium chloride. Experimentally, 25 mL of lysate was diluted with 25 mL of 10 mM sodium phosphates, pH 7.4, containing 10% glycerol and 0.625% Tween-80. The sodium chloride concentration or magnesium chloride concentration was adjusted such that the final concentration in each dilution buffer would be 2× the final salt concentration once the viral containing lysate was diluted 7.5 g of DE was added to the solution. Trace amounts of another metal, preferably zinc, are also present or added to solution. The mixture was stirred at room temperature for 30 min. The solution was then filtered using a 0.45 um CA filter (Gelman Sciences, Ann Arbor Mich., USA). Ten mL of the filtrate was loaded onto an equilibrated Qiagen-500 tip. Assay was performed according to the manufacturer's procedure. Viral recovery was determined by the viral titer assay as described. Results of these experiments are presented in FIG. 3 (sodium chloride optimization) and FIG. 4 (magnesium chloride optimization). As can be seen in the figures optimal salt concentrations may be required for complete separation of DNA and RNA from adenovirus. The optimal salt concentrations are 125 mM and 50 mM for sodium chloride and magnesium chloride respectively.

Viral titer assays, performed on samples taken pre and post filtration, revealed that viral recovery averaged 96%.

Optimization of Virus Separation from DNA during DE Filtration:

Optimal metal ion or salt concentrations may be required for complete separation of DNA and RNA from adenovirus. The metal ion useful for the present invention may be any metal, subject to the provision that metals with known high toxicity should be avoided. Metals which may be suitable for use in the present invention thus include zinc, nickel, barium, iron, copper, cobalt, magnesium, sodium, potassium, and manganese. The salts useful for the present invention may be any acceptable salt form, and would thus include acetate, citrate, sulfate, phosphate, and chloride. Optimal metal ion or salt concentrations may be determined experimentally, as described in the examples below. Such routine experimentation is within the skill of the art. The following are examples of optimal concentrations, for sodium chloride, concentration is preferably in the range of about 75 to about 200 mM for sodium chloride, more preferably about 100 mM to about 150 mM, and most preferably about 125 mM. For magnesium chloride, salt concentration is preferably in the range of about 20 mM to about 100 mM, more preferably about 40 mM to about 75 mM, and most preferably about 50 mM for magnesium chloride. For most metal ions, including zinc, nickel, barium, concentration is preferably in the range of from about trace levels to about 10 mM, more preferably about 0.1 mM to about 0.7 mM, and most preferably from about 0.2 mM to about 0.5 mM. For use of sodium, potassium or magnesium salts, such as sodium chloride or magnesium chloride, it is preferred to also have present or added trace amounts of a metal ion, such as zinc, barium, copper, ferric iron, or nickel. By “trace amounts,” it is meant an amount of metal ion that is above detectable levels, or at least about 1.0 uM.

In addition to, or in place of metal ions, one or more of the following materials may be useful in the methods of the present invention: histidine, imidizole, glysoglycine and thymidine. Also useful in addition to or in place of metal ions may be DNA condensing agents, such as spermine, spermidine, polyethylene glycol, as well as variants of these or other polymers or chemical compounds known to have DNA condensing activity. The inventors also propose that metal chelators [e.g., metal ions] and/or DNA condensing agents, such as described above, may be useful for methods of purification of DNA and/or RNA.

For treatment with diatomaceous earth [DE], preferred concentrations are from about 10 g of DE/L of lysate to about 100 g/L lysate, more preferably from about 30 to about 50 g/L of lysate, and most preferably about 35 to about 45 g/L of lysate.

It is preferred that pH ranges for the methods of the present invention avoid extreme acidity or alkalinity which could disrupt the salt formation. Thus, it is preferred that pH be within a range of from about 5 to about 9, more preferably from about 6 to about 8, and most preferably from about 6.5 to about 7.5.

In some instances it was found that virus could be also be bound to DE. Under these conditions it was found that separation of virus from host cell polynucleotides could be optimized by adjustment of pH, sodium chloride concentration, metal ion concentration or by addition of trace amounts of certain amino acids or amino acid analogs such as histine or imidazole.

Treatment with Diatomaceous Earth (DE).

Example with zinc acetate:

After cell lysis, the virus-containing lysate is diluted by addition of an equal volume of a solution containing 10 mM sodium phosphate buffer pH 7.4 containing 10% Glycerol, 0.25% Tween 80. The mixture is stirred and 5 M zinc acetate is added to the diluted lysate to a final concentration of 0.35 mM. DE (Pharmaceutical-grade CellPure™ P300, Advanced Minerals, Santa Barbara, Calif., USA) is then added to the suspension of cell debris at a ratio of 0.1 g of DE to 2 mL of diluted lysate solution. The combined virus/cell lysate/DE suspension is then stirred at 4° C. for 30 min. to achieve a uniform suspension. While the suspension is stirring, host cell DNA, RNA and other host cell components are allowed to adsorb to the DE. The cell debris and DE are then removed by pumping the suspension through a dead-end Biocap filtration device (CUNO Fluid Purification, Meriden, Conn., USA). (Note: Any type of dead-end or depth filtration device known to the art that allows virus particles to flow through and, at the same time, retains the DE-DNA complex and other cell-associated solids can be used for this step). Note that metal ions other than zinc can be used to promote binding of DNA to DE. Examples of these metals include, but are not limited to, ferric iron, nickel, and barium. The experimental determination of optimal concentrations for these metal ions is within the skilled artisan's ability.

Example with Magnesium Chloride:

After cell lysis the adenoviral containing lysate is diluted by addition of an equal volume of solution containing 10 mM sodium phosphate buffer pH 7.4 containing 10% Glycerol, 0.25% Tween 80, 150 mM MgCl₂, and preferably a trace amount of a metal ion, preferably zinc, is also present. DE (Pharmaceutical grade CellPure™ P300, Advanced Minerals, Santa Barbara, Calif., USA) is added to the suspension of cell debris at an optimized ratio of 0.1 g of DE to 2 mL of diluted lysate solution and stirred at 4° C. for 30 min. While the suspension is stirring, host cell DNA, RNA and other host cell components are allowed to adsorb to the DE. The cell debris and DE are then removed by dead end (or depth) filtration. (Note: Any type of dead-end or depth filtration device known to the art that allows virus particles to flow through and, at the same time, retains other cell-associated solids can be used for this step).

### Example 2

### Optimization of DNA Binding to Whatman™ CDR (CDR)

The virus-containing lysate is diluted by addition of an equal volume of a solution containing 10 mM sodium phosphate buffer containing 10% Glycerol, 0.25% Tween 80. The pH and salt (sodium chloride) concentration of the buffer is adjusted to both maximize binding of host cell polynucleotide contaminants while maximizing recovery of virus. CDR (Cell Debris remover, Whatman™ Biochemicals, Maidstone, England) is then added to the suspension at a ratio of 0.1 g CDR/mL of solution. The combined virus/cell lysate/CDR suspension is then stirred at 4° C. for 30 min. to achieve a uniform suspension. The virus containing solution is separated from the filter aid and cellular debris using any type of filter known to the art or by centrifugation.

Filtration using Poly-anionic Cellulose

As an alternative to DE filtration the virus-containing lysate was diluted at a one-to-one ratio with 10 mM sodium phosphate buffer pH 7.4 containing 10% glycerol, 0.25% Tween 80. The sodium chloride concentration was adjusted so that virus would flow through the filtration device into the filtrate while the host cell nucleotides would be retained with the filter aid and cell debris. CDR (Cell Debris remover, Whatman™ Biochemicals, Maidstone, England) was then added to the suspension at a ratio of 0.1 g CDR/mL of solution. The combined virus/cell lysate/CDR suspension was then stirred at 4° C. for 30 min. to achieve a uniform suspension. While the suspension was stirring, host cell DNA, RNA and other host cell components were allowed to adsorb to the CDR. The cell debris and DE were then removed by pumping the suspension through a dead-end Biocap filtration device (CUNO Fluid Purification, Meriden, Conn., USA). The CDR filtrate containing the Ad2 virus was collected for further processing.

For treatment with CDR, preferred concentrations are from about 10 g of CDR/L of lysate to about 100 g/L lysate, more preferably from about 30 to about 50 g/L of lysate, and most preferably about 35 to about 45 g/L of lysate.

(Note: Any type of dead-end or depth filtration device known to the art that allows virus particles to flow through and, at the same time, retains the CDR-DNA complex and other cell-associated solids can be used for this step).

### Example 3

### Adenovirus Purification Process Using Diatomaceous Earth (DE) Filtration

Diatomaceous Earth (DE) Filtration

In place of a dilution using 10 mM phosphate buffer as in Example 1, the cell lysate containing adenovirus, serotype 2 (Ad2) was diluted at a one-to-one ratio with 10 mM Tris buffer, pH 7.3, containing 10% glycerol, 0.25% Tween 80. After dilution, 5 M zinc chloride stock was added to a final concentration of 0.35 mM. After stirring, DE was added to the lysate at a ratio of 40 g DE per L of lysate and stirred for 15 to 30 min. to achieve a uniform suspension. Cell debris and DE (with cellular components adsorbed) were retained by pumping the suspension through a dead-end filtration system. The filtrate (containing the Ad2 virus that was not adsorbed to DE) was collected for further processing.

Alternatively, the cell lysate was diluted with phosphate-buffered saline (PBS), pH 7.3, containing 10% glycerol, 0.25% Tween 80, 150 mM MgCl₂ and trace amounts of zinc ion, at a ratio of 1L of buffer to 1L of lysate. After stirring to achieve a uniform suspension, DE was added to the lysate at a ratio of 40 g DE per L of lysate and the resulting suspension was stirred for 15 to 30 min. Cell debris and DE were retained by pumping the suspension through a Biocap (CUNO Fluid Purification, Meriden, Conn., USA) filtration device. The DE filtrate (containing the Ad2 virus) was collected.

Tangential Flow Filtration (TFF)

The resulting DE filtrate was concentrated using an AG/T UFP 500 C9A TFF device fitted with a membrane having a nominal molecular weight (NMW) cutoff of 500,000 Daltons (500 kD). In this step, the filtrate (containing Ad2 virus) was first concentrated between 4- and 8-fold by ultrafiltration (UF). The retained concentrate (retentate) was then dialyzed or diafiltered (DF) against 7- to 10-volumes of a suitable chromatography buffer (e.g., phosphate or Tris at pH 6 to 8 respectively).

Chromatography

(Note: Although in this example pseudo-affinity chromatography or hydrophobic interaction chromatography is used prior to anion or cation exchange chromatography, the order can be altered or rearranged under appropriate manipulation of buffer conditions; and other chromatographic techniques familiar to those knowledgeable in the art can be used.)

A) Pseudo-affinity Chromatography

A column of Mimetic Blue 1 A6XL resin was equilibrated in PBS, pH 7.3, containing 10% glycerol, 0.25% Tween 80 (equilibration buffer). The DF retentate was then loaded onto the column at a linear flow rate of 50 cm/hr. The column was washed with equilibration buffer containing 20 mM sodium chloride and the virus was subsequently eluted with equilibration buffer containing 0.2 M sodium chloride.

(Note: Examples of, but not restricted to, pseudo-affinity resins appropriate for purification of adenoviruses include Mimetic Blue (1 and 2) A6XL, Mimetic RED (2 and 3) A6XL, Mimetic Orange (1, 2 and 3) A6XL, Mimetic Yellow (1 and 2) A6XL and Mimetic Green A6XL (ProMetic Biosciences, Montreal (Quebec) Canada), and Blue Sepharose CL-6B and Red Sepharose CL6B (AmershamPharmacia Biotech, Upsala, Sweden.)

B) Hydrophobic Interaction Chromatography (HIC)

A column of EMD Phenyl resin was equilibrated in PBS, pH 7.3, containing 10% glycerol, 0.25% Tween 80 and 0.25 M (NH₄)₂SO₄ (HIC Buffer). The DF retentate was diluted in a volume ratio, 1:1 with 2× salt HIC Buffer (PBS, pH 7.3, containing 10% glycerol, 0.25% Tween 80 and 0.5 M (NH₄)₂SO₄) and then loaded onto a column at a linear flow rate of 50 cm/hr. In this, the preferred embodiment, the virus particles are not adsorbed to the HIC resin, and particles are recovered in the non-adsorbed, (i.e., unbound) and wash fractions leaving contaminants bound to the column. The column flow through, containing the virus, was collected (HIC Pool) for further processing.

Note: Adenovirus can also be adsorbed to the HIC resin. Under conditions where the virus particles are adsorbed to the resin, particles (after an appropriate column wash step) can be recovered in a low salt elution step. Under these conditions, portions of the contaminants are distributed in the flow through and wash and others, under appropriate conditions, remain adsorbed to the resin after the virus particles have been removed.

(Note: Examples of, but not restricted to, HIC resins appropriate for purification of adenoviruses include EMD phenyl and EMD propyl (EM Separations Technology, Gibbstown, N.J., USA) or Phenyl and Octyl Sepharose (AmershamPharmacia Biotech, Upsala, Sweden) or TSK ether, butyl and phenyl (TosoHaas, Montgomeryville, Pa., USA).

C) Anion Exchange Chromatography (AEX)

An AEX column containing EMD DEAE resin was equilibrated in AEX buffer (phosphate-buffered saline (PBS), pH 7.3, containing 10% glycerol, 0.25% Tween 80, plus additional 0.1 M NaCl and 0.1 M KCl). The HIC Pool was then loaded onto the AEX column. Under this condition, virus particles and contaminating proteins adsorbed to the resin. The contaminating protein was removed by washing the column with AEX Wash Buffer (PBS, pH 7.3, containing 10% glycerol, 0.25% Tween 80, plus additional 0.16 M NaCl and 0.16 M KCl). To remove and collect the virus particles, the column was eluted with AEX Elution Buffer (PBS, pH 7.3, containing 10% glycerol, 0.25% Tween 80, plus additional 0.19 M NaCl and 0.19 M KCl). Purified virus appearing in the AEX elution was collected and stored at −80° C. until formulation. (Note: Examples of, but not restricted to, appropriate anion exchange resins include EMD DEAE (EM Separations Technology, Gibbstown, N.J., USA) or DEAE Sepharose (AmershamPharmacia Biotech, Upsala, Sweden) or TSK DEAE 650 and 750 (TosoHaas, Montgomeryville, Pa., USA.)

D) Cation Exchange Chromatography (AEX)

A column of EMD CE resin was equilibrated in PBS, pH 7.3, containing 10% glycerol, 0.25% Tween 80 (equilibration buffer). The HIC pool was diluted 5 fold with equilibration buffer then loaded onto the column at a linear flow rate of 50 cm/hr. The column was washed with equilibration buffer and then subsequently eluted with equilibration buffer containing 0.5 M sodium chloride. (Note: Examples of (but not restricted to) appropriate cation exchange resins include: EMD SO₃ and EMD COO (EM Separations Technology, Gibbstown, N.J., USA), CM and S Sepharose (AmershamPharmacia Biotech, Upsala, Sweden) and TSK CM and SP (TosoHaas, Montgomeryville, Pa., USA)

### Example 4

### Adenovirus Purification Process Using Whatman™ CDR Filtration

Filtration using Whatman™ CDR

The virus-containing lysate is diluted by addition of an equal volume of a solution containing 10 mM sodium phosphate buffer pH 7.0 containing 10% Glycerol, 0.25% Tween 80 and 0.52 M sodium chloride. CDR (Cell Debris remover, Whatman™ Biochemicals, Maidstone, England) is then added to the suspension at a ratio of 0.1 g CDR/mL of solution. The combined virus/cell lysate/CDR suspension is then stirred at 4° C. for 30 min. to achieve a uniform suspension. The virus containing solution is separated from the filter aid and cellular debris using any type of filter known to the art or by centrifugation.

Further Purification of Adenovirus

After filtration, the virus was purified by methods similar to those described in Example 3.

TABLE 1
Concentrations of Metal Salts Tested for Optimal Binding of DNA to DE

| Nickel | Zinc | Ferric Iron | Barium |
|---|---|---|---|
| Chloride | Chloride | Chloride | Chloride |
| mM | mM | mM | mM |
| 0 | 0 | 0 | 0 |
| 0.5 | 0.5 | 1 | 1 |
| 1 | 1 | 5 | 2 |
| 2.5 | 5 | 10 | 10 |
| 5 | 10 | 15 | 15 |
| 7.5 | 15 | 25 | 25 |
| 10 | 25 | 75 | 75 |
| 15 | 75 | 150 | 150 |
| 25 | 150 | | |
| 75 | | | |
| 150 | | | |

TABLE 2
Concentrations of salt (MgCl₂ or NaCl) tested for optimal binding of DNA
contamination to DE

| MgCl2 | NaCl |
|---|---|
| concentration | concentration |
| (mM) | (mM) |
| 0 | 0 |
| 25 | 25 |
| 50 | 50 |
| 75 | 75 |
| 125 | 125 |
| 250 | 250 |
| | 500 |

DNA binding as a function of pH was also investigated. These data are reported in Table 3. Experimentally the pH of 25 mL of lysate was adjusted to either pH 8 or pH 6 by addition of either dibasic sodium phosphate of monobasic sodium phosphate respectively. DE filtration was performed on each aliquot as described above. A control aliquot, where no pH adjustment was made, was also DE filtered. As can be seen as binding of DNA to DE was pH dependent. At higher pH more DNA bound to DE than at lower pH. When viral titer assays were performed on the DE filtrates, however, a decrease in viral recovery was noted when the pH of the unit operation was raised.

TABLE 3
DNA removal as a function of pH

| Sample | % Removal |
|---|---|
| pH 8 | 77.2 |
| pH 7.4 | 59.1 |
| pH 6 | 29.1 |

As can be seen increasing pH results in greater removal of DNA while decreasing pH inhibits removal. It should be noted that viral recovery in DE filtrates decreases with increasing pH as determined by the viral titer assay.

The disclosure of all of the publications cited within are hereby incorporated by reference.

Many modifications and alterations to the above reagents, materials and procedures are contemplated, and are within the skill of the art. Thus, these modifications and alterations comprise part of the invention. It is within the capability of the skilled artisan to recognize that the above reagents, resins and other analogous materials may be used, as appropriate, for the purification of other encapsulated viruses, such as adeno-associated virus, alphaviruses, herpes simplex viruses, and other retroviruses, such as lentiviruses.

We claim:

1. A method for purification of encapsulated viruses from cell culture, said method comprising:
(a) lysing a cell culture containing encapsulated virus;
(b) subjecting the composition resulting from step (a) to filtration with a substance selected from the group consisting of Diatomaceous Earth (DE) and poly-anionic cellulose filter aids, to generate a filtrate;
(c) subjecting the filtrate of step (b) to one or more suitable concentration and diafiltration steps to generate concentrated and diafiltered retentate; and
(d) subjecting the concentrated or diafiltered retentate of step (c) to one or more suitable purification steps and collecting a purified composition containing encapsulated viruses, wherein steps (c) and steps (d) may take place iteratively.

2. The method of claim 1, wherein step (b) comprises the use of optimal concentrations of metal ion salts during DE filtration to promote maximal DNA and RNA binding.

3. The method of claim 2, wherein the metal ion salt is a metal ion salt of a metal selected from the group consisting of zinc, nickel, ferric, copper, barium, magnesium manganese, sodium, cobalt or potassium.

4. The method of claim 1, wherein step (b) comprises the use of optimal concentrations of metal ion salts during DE filtration to promote maximal DNA and RNA binding and the use of histidine, imidazole or another amino acid that can modify the binding of metal ions to either the host cell nucleotides or to the virus.

5. The method of claim 1 wherein step (b) comprises the use of a poly-anionic cellulose based filter aid.

6. The method of claim 5 wherein salt concentration is adjusted so that host cell nucleotides bind to poly-anionic celluloses and virus flows through the filter into the filtrate.

7. The method of claim 3, wherein step (c) comprises loading the filtrate from step (b) into a device used for concentration of biological molecules.

8. The method of claim 3, wherein step (c) comprises employing a dialysis or buffer-exchange device which device comprises a membrane having a pore size suitable for retaining virus particles.

9. The method of claim 3, wherein step (c) comprises employing a dialysis or buffer-exchange device which device comprises a resin having a pore size capable of separating the virus particles from larger and smaller molecular size contaminants.

10. The method of claim 3, wherein step (c) comprises employing a concentration device which device comprises a membrane pore size suitable for the passage of materials containing molecular sizes smaller than virus particles.

11. The method of claim 8, wherein said process further comprises concentrating the retained virus particles are concentrated in solution by ultrafiltration.

12. The method of claim 9, wherein step (c) comprises diafiltering the composition containing virus particles to produce a composition containing diafiltered, non-concentrated virus particles prior to concentration.

13. The method of claim 3, wherein step (c) comprises concentrating the composition containing virus particles to produce a composition containing concentrated, non-diafiltered virus particles prior to diafiltration.

14. The method of claim 12, wherein the diafiltration step of step (c) is suitable for loading the diafiltered, non-concentrated virus particles onto a suitable anion exchange chromatography resin.

15. The method of claim 12, wherein the diafiltration step of step (c) is suitable for loading the diafiltered, non-concentrated virus particles onto a suitable hydrophobic interaction chromatography resin to generate a flow-through pool.

16. The method of claim 12, wherein the diafiltration step of step (c) is suitable for loading the diafiltered, non-concentrated virus particles onto (and promoting their adsorption to) a suitable pseudo-affinity resin.

17. The method of claim 12, wherein the diafiltration step of step (c) is suitable for loading the diafiltered, non-concentrated virus particles onto (and promoting their adsorption to) a suitable cation exchange chromatography resin.

18. The method of claim 12, wherein the diafiltration step of step (c) is suitable for loading the diafiltered, concentrated virus particles onto (and promoting their adsorption to) a suitable pseudo-affinity resin.

19. The method of claim 10, wherein the concentrated and diafiltered filtrate of step (c) is suitable for mixing the concentrated virus particles with cesium chloride.

20. The method of claim 10, wherein the concentrated and diafiltered filtrate of step (c) is suitable for loading the concentrated virus particles onto (and promoting their adsorption to) a suitable anion exchange chromatography resin.

21. The method of claim 13, wherein the concentration step of step (c) is suitable for loading the concentrated, non-diafiltered virus particles onto a suitable hydrophobic interaction chromatography resin to generate a flow-through pool.

22. The method of claim 13, wherein the concentration step of step (c) is suitable for loading the concentrated, non-diafiltered virus particles onto (and promoting their adsorption to) a suitable cation exchange chromatography resin.

23. The method of claim 3, wherein the purification steps of step (d) comprise mixing the concentrated virus particles with cesium chloride and subjecting the mixture to ultracentrifugation.

24. The method of claim 12, wherein the purification steps of step (d) comprise first loading the composition containing diafiltered, non-concentrated virus particles of claim 12 onto and adsorbing the encapsulated virus to a suitable anion exchange chromatography column and using suitable elution to collect a purified composition containing encapsulated viruses.

25. The method of claim 20 wherein the purification steps of step (d) comprise loading the concentrated and diafiltered filtrate of step (c) containing encapsulated viruses onto a suitable anion exchange chromatography resin to produce a purified composition containing encapsulated viruses, followed by a second purification step of loading the purified composition containing encapsulated viruses onto a suitable hydrophobic interaction chromatography resin under conditions to generate a flow-through pool and collecting a purified composition containing encapsulated viruses in the flow-through pool.

26. The method of claim 20, wherein the purification steps of step (d) comprise loading the concentrated, non-diafiltered virus particles of step 13 onto a suitable hydrophobic interaction chromatography resin to generate a flow-through pool, followed by loading the flow-through pool onto a suitable cation exchange resin and, using suitable elution conditions, and collecting a purified composition containing encapsulated viruses.

27. The method of claim 1 wherein the purification steps of step (d) comprise adsorbing the concentrated and diafiltered filtrate of step (c) to a suitable anion exchange chromatography resin and, using suitable elution conditions, collecting a purified composition containing encapsulated viruses.

28. The method of claim 23 wherein the purification steps of step (d) further comprise loading the purified composition containing encapsulated viruses of claim 23 onto a suitable hydrophobic interaction chromatography column under conditions to generate a flow-through pool and collecting a purified composition containing encapsulated viruses in that flow-through pool.

29. The method of claim 24 wherein the purification steps of step (d) further comprise adsorbing the purified composition containing encapsulated viruses from the flow-through pool of claim 24 to a suitable cation exchange resin and, using suitable elution conditions, collecting a purified composition containing encapsulated viruses.

30. The method of claim 25, wherein the purification steps of step (d) further comprise adsorbing the purified composition containing encapsulated viruses of claim 25 to a suitable cation exchange resin and, using suitable elution conditions, collecting a purified composition containing encapsulated viruses.
